(19) United States
(12) Patent Application Publication
(10) Pub. No.: US 2009/0042958 A1
(43) Pub. Date: Feb. 12, 2009

(54) Composition and method for treating immune-mediated skin disorders

(75) Inventor: Jiajiu Shaw, Ann Arbor, MI (US)

Correspondence Address: BUTZEL LONG;IP DOCKETING DEPT, 350 SOUTH MAIN STREET, SUITE 300, ANN ARBOR, MI, 48104, US

(73) Assignee: Geneblue Corporation, Los Angeles, CA (US)

(21) Appl. No.: 11/899,095

(22) Filed: Sep. 4, 2007

Related U.S. Application Data
(60) Provisional application No. 60/842,293, filed on Sep. 5, 2006.

(51) Int. Cl. A61K 31/42; C07D 261/18; A61K 31/167; C07C 237/00; A61P 17/00

(52) U.S. Cl. 514/378; 548/248; 514/626; 564/194

(57) ABSTRACT

Liquid or semi-liquid pharmaceutical compositions for topically treating immune-mediated skin disorders such as psoriasis, eczema, acne, dermatitis, and skin cancer and methods of treating patients having such immune-mediated skin disorders using the compositions.

### RELATED APPLICATION

This application is based upon U.S. Provisional Patent Application Ser. No. 60/842,293, filed Sep. 5, 2006, and claims priority thereto under 35 U.S.C. §120.

### TECHNICAL FIELD

The present invention relates to compositions and methods for treating immune-mediated skin disorders.

### BACKGROUND ART

Psoriasis is a chronic skin disorder marked by periodic flare-ups of sharply defined red patches covered by a silvery, flaky surface; the primary disease activity leading to psoriasis occurs in the epidermis.

Even in its mildest form, psoriasis can cause itching, burning, stinging, and bleeding. These symptoms can be very debilitating in more severe cases. The severity of psoriasis ranges from one or two flaky inflamed patches to widespread pustular psoriasis that, in rare cases, can be life threatening.

### Treatment Options

In general, the following three traditional treatment options, which are listed as from least to the greatest potency, are available for treating psoriasis:
Topical Medications: Options include lotions, ointments, creams, and shampoos. These may be useful for mild-to-moderate psoriasis. However, currently available topical medicines rarely produce complete clearance. Examples of topical drugs are alclometasone dipropionate cream and ointment (by GlaxoSmithKline), Psoriatec cream (by Sirius), and Temovate cream and gel (by GlaxoSmithKline).
Phototherapy: Options include light-wave radiation treatments using broad or narrow band ultraviolet B (UVB) or psoralen with ultraviolet A (PUVA). This therapy appears to be effective for moderate-to-severe psoriasis.
Systemic Oral Drugs: This treatment employs various oral drugs that affect the whole body system, not just the skin. These agents have significant side effects and are generally reserved for severe psoriasis, particularly when more than 10 percent of the body is involved. Examples are cyclosporin and methotrexate.

Abnormal expression of inflammatory mediators or their receptors in keratinocytes are relevant to the pathogenesis of chronic inflammatory skin disorders such as psoriasis, atopic dermatitis and allergic contact dermatitis. Research over the last several years has led scientists to be increasingly convinced that psoriasis is caused by overactive or faulty cells in the immune system. In particular, cytokines secreted by the immune system are thought to be responsible for several symptoms of psoriasis.

Regulating the secretion or inhibiting the activities of cytokines has become one of the most promising answers in the search for a cure of psoriasis.

### TNF-α

TNF-α (tumor necrosis factor-α) is an important cytokine. Similar to other cytokines, when it is overproduced, TNF-α can cause inflammation. In people with psoriasis, TNF-α is found to be overabundant in the epidermis.

Under normal circumstances when there are no antigens to fight, the body produces soluble TNF-α receptors that inactivate TNF-α before it can attach to the immune cells. This natural process is somehow sabotaged in people with psoriasis since the immune system continues to produce large amounts of TNF-α as if an antigen continues to be present while the body creates insufficient numbers of soluble TNF-α receptors.

### Biologics

A relatively new type of therapeutics is known as biologics, which focuses on immune modulating medications. Biologics are made up of protein molecules derived from living sources such as viruses, animals, and people.

The FDA (Food and Drug Administration) approved a new biologic in April, 2004 for a new use of Enbrel® (etanercept) to treat moderate to severe plaque psoriasis. Acting similarly to a TNF-α receptor, etanercept specifically targets TNF-α and captures and inactivates excess TNF-α and thereby interrupts the chain of events that leads to the immune-mediated diseases. Another biologic, Remicadeg (infliximab) is being prescribed “off-label” for psoriatic arthritis. Infliximab also targets and blocks TNF-α. However, infliximab is not used alone and is sometimes prescribed in combination with an immunosuppressive, methotrexate.

Briefly, entercept and infliximab are effective for treating psoriasis, indicating that TNF-α therapy is effective. However, entercept and infliximab are associated with several obvious problems:
They must be injected, and are thus inconvenient.
They are expensive to make due to the nature of the products (protein molecules)
They pose potentially serious adverse effects, such as tuberculosis, because they block the activity of TNF-α excessively.

Therefore, a topically administered small molecule TNF-α modulator would provide a significant improvement over the most advanced drugs that are currently being used, i.e., entercept and infliximab. However, prior to the present invention there was not liquid or semi-liquid composition comprising small molecule TNF-α modulator as a topical drug for treating psoriasis and other immune-mediated skin disorders.

### DISCLOSURE OF THE INVENTION

According to various features, characteristics and embodiments of the present invention which will become apparent as the description thereof proceeds, the present invention provides a liquid or semi-liquid pharmaceutical composition that includes one of more of the following components: (1) UTL-5, (2) UTR-1, (3) a compound of the formula (I), and (4) a compound of the formula (II):

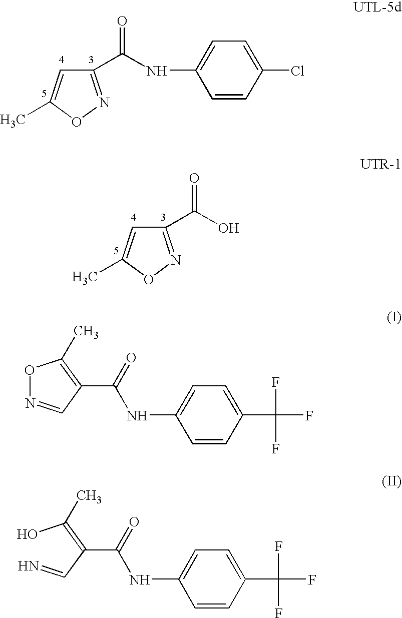

The compositions can be provided in the form of various liquids or semi-liquids such as solutions, emulsions, suspensions, gels, creams, lotions, and can be administered or applied in any suitable fashion including incorporation into transdermal patches.

The compositions can be used in methods that treat patients with an immune-mediated skin disorders by administering a therapeutically effective amount of the composition(s) topically. Immune-mediated skin disorders that can be treated are exemplified by psoriasis, eczema, acne, dermatitis, and skin cancer, including basal cell carcinoma.

### BEST MODE FOR CARRYING OUT THE INVENTION

It has been known that some isoxazole derivatives are TNF-α modulators. A specific example is compound (I), 5-methylisoxazole-4-carboxylic acid-(4-trifluoromethyl)-anilide.

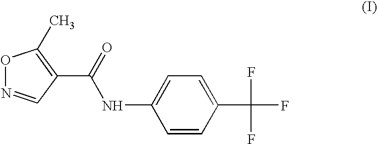

Studies conducted on treating psoriasis with compound (I) have been reported (K. Reich et al., British Journal of Dermatology, 2002, 146, 335-336); however, there are no reports of topical application of compound (I).

A solid composition of compound (I) was patented (U.S. Pat. No. 7,071,222) for treating autoimmune diseases including rheumatoid arthritis, systemic lupus erythrematosus, multiple sclerosis, and psoriasis. However, there has been no report as to a liquid or semi-liquid composition of compound (I) and its topical administration for treating psoriasis.

The present inventor recently developed a novel in vitro model (U.S. patent application Ser. No. 11/343,835), which employs EpiDerm™ to screen agents for treating psoriasis. Using this model, it can be shown, according to the present invention, that several isoxazole derivatives are effective in reducing TNF-α released from commercially available skin tissue model, EpiDerm™, which comprises normal, human-derived epidermal keratinocytes cultured to form a multilayered, highly differentiated model of the human epidermis. During the course of the present invention it was discovered that these isoxazole derivatives are able to penetrate into EpiDerm™ tissues and reduce TNF-α, and thus can be used as topical agents. This finding is unique because there has been no report clearly indicating that TNF-α is significantly induced from EpiDerm™ tissues. Furthermore, there has been no report on using isoxazole derivatives to apply on top of EpiDerm™ to reduce TNF-α released from EpiDerm™ tissues.

### EXAMPLE

### Reduction of TNF-α from EpiDerm™ Tissues In Vitro by Isoxazole Derivative

### Test Materials:

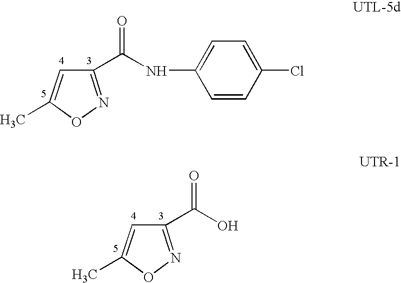

Stock solutions of test materials are listed below:
1. UTL-5d stock: 1.7 mg/ml UTL-5d in a vehicle of 50:50 EtOH:PEG 600 v/v
2. UTR-1 stock: 3.5 mg/ml UTR-1 in a vehicle of 50:50 EtOH:PEG 600 v/v
3. Vehicle stock: 50:50 EtOH:PEG 600

### Pretreatment

Human epidermal keratinocytes were seeded into 6-well plates and grown at 37±2° C. and 5±1% CO₂ using serum free Epilife media supplemented as recommended by the manufacturer. Upon reaching confluency, the media were removed and the cells were treated overnight with Epilife media containing 1% v/v each of the stock solutions above (Vehicle stock and UTR-1 stock). Final concentration was 35 μg/ml for UTR-1. Two wells in the 6-well plate were prepared for each treatment. After applying the test material the cells were incubated for 24 hours at 37±2° C. and 5±1% CO₂.

### Treatment and Micro Array Analysis

At the end of the incubation period the culture media was removed via aspiration and replaced with phosphate buffered saline. The cells were then irradiated with ˜30 mJ/cm² of UVB radiation. Fresh culture media was then applied to the cells and the cells were incubated overnight at 37±2° C. and 5±1% CO₂. After the incubation the cell culture media was collected to measure TNF-α release by a commercial ELISA Assay kit.

The cells were washed once with PBS and then a trypsin/EDTA solution was added to release the cells, followed by the addition of trypsin neutralizing solution. The cells were collected and pooled into 15 ml centrifuge tubes based on their treatment and pelleted by centrifuging at 1000 RPM at 4±2° C. After removing the supernatant, the pelleted cells were lysed by adding 500 μl of guanidinium thiocyanate lysis solution to each tube and then repeatedly drawing and releasing the solution into the pipette until the cell pellet is dissolved. Total RNA was then isolated by an RNAqueous kit (Ambion). After purifying the RNA, mRNA is isolated and the converted into anti-sense RNA (aRNA). The aRNA is then labeled with a florescent probe. In this case, Cy3 (green florescent signal) is used to label the sRNA from untreated sample, while Cy5 (red fluorescent) is used to label the aRNA of treated sample. Once the aRNA is labeled and any unincorporated dye is removed from the sample, the labeled aRNA is mixed with a hybridized solution and applied to the microarray. The microarray is then hybridized overnight at 60-65° C. After hybridization, the microarray is washed to remove any unbound aRNA probe and then scanned with a microarray scanner. Criteria for evaluating changes in gene expression may vary. In general, (1) the fluorescence intensity of the gene marker should be greater than the background intensity, and (2) the ratio of Cy5/Cy3 (treated/untreated) fluorescence intensity needs to be greater than 1.3 or less than 0.66 to indicate a change of ±30% in gene expression.

### Results:

As shown in the following table, the results show that both isoxazole derivatives are able to penetrate into EpiDerm™ tissues and reduced elevated levels of TNF-α.

| | Abs | Abs | TNF-α | TNF-α | | |
|---|---|---|---|---|---|---|
| | sample | sample | (pg/ml), | (pg/ml), | | |
| Treatment | 1 | 2 | sample1 | Sample2 | Avg. | Stdev |
| UVB + Vehicle | 0.304 | 0.172 | 257 | 147 | 202 | 78 |
| UVB + UTL5-d | 0.069 | 0.076 | 61 | 67 | 64 | 4 |
| UVB + UTR-1 | 0.140 | 0.139 | 120 | 119 | 120 | 1 |

Gene array analysis results indicate that a number of gene expressions were suppressed (relative to background). The genes of interest include JAK3, MAP3K2, and cancer related genes. The follow table shows the suppression of JAK3 and MAP3K2 genes:

| Gene | UTL-5d | UTR-1 |
|---|---|---|
| Name | Gene expression (%) | Gene expression (%) |
| JAK3 | 34.7 | 38.6 |
| JAK3 | 29.3 | 41.7 |
| MAP3K2 | 59.9 | 59.2 |

The following table shows the suppression of colon cancer and TNF-α related genes:

| | UTL-5d | UTR-1 |
|---|---|---|
| Gene Name | Gene expression (%) | Gene expression (%) |
| APOBEC1 | 29.2 | 33.3 |
| CDCP1 | 44.1 | 65.6 |
| DEFA6 | 44.3 | 38.0 |
| EFA6R | 29.2 | 37.5 |
| EPHB4 | 35.8 | 43.3 |
| ITGB6 | 24.3 | 30.0 |
| PTGER1 | 33.8 | 28.6 |
| PTGER2 | 39.9 | 58.0 |
| PTGER3 | 42.7 | 40.8 |
| PTGFR | 24.5 | 40.0 |
| PTGIR | 27.0 | 39.4 |
| TGFB2 | 38.2 | 57.9 |
| TNFRSF6B | 52.1 | 68.0 |
| TNFSF13 | 47.0 | 42.5 |
| TUCAN | 26.0 | 33.3 |

The present invention provides a liquid or semi-liquid pharmaceutical composition comprising one or a plurality of (1) UTL-5d, (2) UTR-1, (3) compound with the formula (I), or (4) compound with the formula (II) shown below:

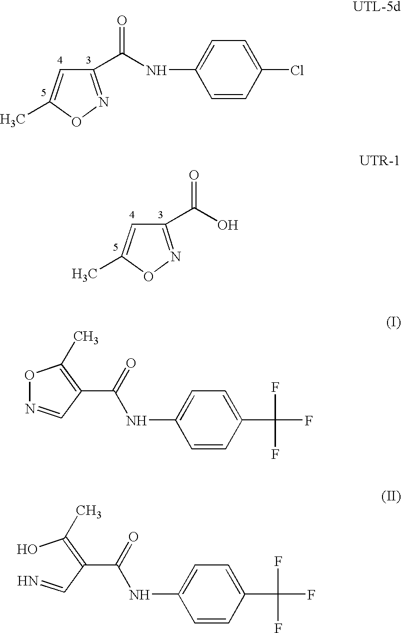

The liquid or semi-liquid composition can be in form of a solution, an emulsion, a suspension, a gel, a cream, a lotion, a transdermal patch or any other convenient form.

The liquid or semi-liquid composition comprises pharmaceutically acceptable excipients. Suitable excipients include water, ethanol, isopropyl alcohol, stearyl alcohol, lanolin alcohol, glycerine, mineral oil, hydrogenated oil, surfactants, propylene glycol, polyethylene glycol, methoxypolyethylene glycol, propylene oxide, poly(ethylene)oxide, methylcellulose, hydroxypropyl methylcellulose, sorbitol, silica, etc. In addition other conventional ingredients such as defoamers, preservatives, fragrances, coloring agents, and the like can be included in the topical compositions.

The present invention also provide a method of treating a patient with immune-mediated skin disorder which method involves administering topically to a patient a therapeutically effective amount of the liquid or semi-liquid pharmaceutical composition, wherein the skin disorders include psoriasis, eczema, acne, dermatitis, and skin cancer.

Basal cell carcinoma, which can be treated according to the present invention, is the most common form of skin cancer. Elevated TNF-α levels are closely associated with basal cell carcinoma.

Although the present invention has been described with reference to particular means, materials and embodiments, from the foregoing description, one skilled in the art can easily ascertain the essential characteristics of the present invention and various changes and modifications can be made to adapt the various uses and characteristics without departing from the spirit and scope of the present invention as described above and as set forth in the attached claims.

What is claimed is:

1. A liquid or semi-liquid pharmaceutical composition comprising one or a plurality of (1) UTL-5, (2) UTR-1, (3) compound with the formula (I), or (4) compound with the formula (II) shown below:

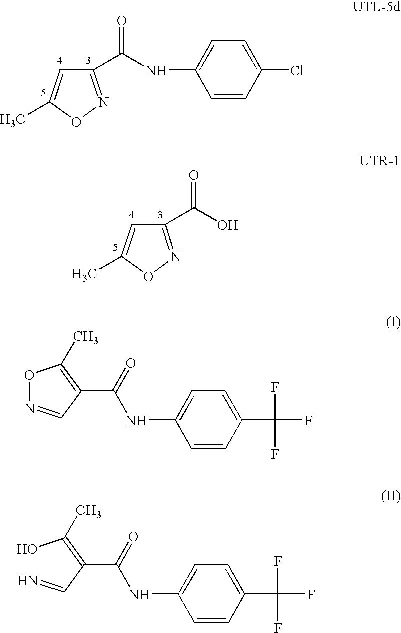

2. The pharmaceutical composition in claim 1, wherein the liquid or semi-liquid composition comprises a solution, an emulsion, a suspension, a gel, a cream, a lotion, or a transdermal patch.

3. A method of treating a patient with an immune-mediated skin disorder comprising administering topically to a patient a therapeutically effective amount of the composition in claim 1.

4. The method of claim 3, wherein the skin disease comprises at least one of psoriasis, eczema, acne, dermatitis, and skin cancer.

5. The method in claim 4, wherein skin cancer comprises basal cell carcinoma.
